(19) United States
(12) Patent Application Publication
(10) Pub. No.: US 2008/0213909 A1
(43) Pub. Date: Sep. 4, 2008

(54) PHARMACEUTICAL PACKAGING ASSAY

(76) Inventors: Daniel E. Haines, Lake Ariel, PA (US); Rajendra J. Redkar, Clarks Summit, PA (US); Luis A. Burzio, Wentzville, MO (US); Samuel D. Conzone, Clarks Summit, PA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/857,636

(22) Filed: Sep. 19, 2007

Related U.S. Application Data
(60) Provisional application No. 60/845,534, filed on Sep. 19, 2006.

(51) Int. Cl. G01N 33/00

(52) U.S. Cl. 436/86

(57) ABSTRACT

A pharmaceutical packaging assay for the determination of drug adsorption to the packaging surface(s) is described. The assay described utilizes fluorescent labeling of drugs in various formulations to determine the best packaging surface for drug stability as a function of adsorption.

This application claims the benefit of the filing date of U.S. Provisional Application Ser. No. 60/845,534, filed Sep. 19, 2006, which is incorporated by reference herein.

Small molecule and biomolecular based drugs, particularly proteins, have a strong attraction for most packaging surfaces that they come into contact. In the pharmaceutical packaging industry this affinity often results in the loss of the valuable drug, resulting in the need for overfilling to counteract the losses due to adsorption, denaturation, and/or agglomeration caused by the interaction with the surface. Much research has been performed to develop surfaces that resist biomolecular adsorption, but due to the ubiquitous nature of biomolecules, no one solution has been found. For proteins, this is largely due to the various factors that influence protein loss through adsorption, denaturation, and agglomeration—solution pH, surface chemistry, surface energy, surface charge, concentration, etc. Thus in the development of new biomolecular based drugs there is the need for analytical methods to directly assay the effects of packaging material on the stability of the packaged drug formulation.

The most common analytical techniques for determining drug adsorption take advantage of the change in optical and/or electrical properties of a surface that has adsorbed proteins. These techniques provide a measurement of the presence/absence of species on a surface. Some techniques allow determination of additional information as to the amount or thickness of adsorbed protein (SPR; ellipsometry; QCM; XPS; radioactive isotopic labeling; solute depletion; fluorescence emission spectroscopy), conformation (ATR FT-IR; Raman scattering; XPS; low angle X-ray reflectivity; scanning force microscopy), or binding energy to the surface (scanning force microscopy). Surface plasmon resonance (SPR) is very sensitive to changes in the index of refraction at and near the surfaces of metal films. SPR can measure the before/during/after protein adsorption to determine kinetic and thermodynamic information regarding the adsorption of proteins. See, for example, Jennifer M. Brockman, Anthony G. Frutos, Robert M. Corn- J. Am. Chem. Soc. 1999, 121, 8044-8051. Ellipsometry can be used to determine if proteins have adsorbed to a surface by measuring the change in the index of refraction before/after protein adsorption to give an experimental thickness of the layer of proteins adsorbed. This detection method is useful if a substrate has a refractive index different from the coating. See, for example, Delana A. Nivens, David W. Conrad-Langmuir 2002,18, 499-504; M. Mrksich, L. E. Dike, J. Tien, D. E. Ingber, G. M. Whitesides—Exp. Cell Res. 1997, 235, 305-313; and Kevin L. Prime, George M. Whitesides—J. Am. Chem. Soc. 1993, 115, 10714-10721. Quartz crystal microbalance (QCM) measures changes in the fundamental frequency of vibration for a quartz crystal for protein adsorption via the piezoelectric effect, yielding adsorbed protein layer thickness. Surface acoustic wave (SAW) and acoustic plate mode (APM) devices takes advantage of changes in surface acoustic waves (velocity and amplitude) when proteins adsorb to the surface of a crystal modified with electrodes, detecting the presence or absence of protein binding. See, for example, Robert Ros Seigel, Philipp Harder, Reiner Dahint, Michael Grunze, Fabien Josse—Anal. Chem. 1997, 69, 3321-3328). X-ray photoelectron spectroscopy (XPS) uses X-rays to eject electrons from atoms; each atom has different XPS spectrum and allows determination of the number and type of atoms per unit area. XPS can also be used to determine if protein has adsorbed to a surface by measuring the spectrum from a protein adsorbed to a surface vs. a non-protein adsorbed surface. Attenuated total internal reflectance Fourier transfer infrared (ATR FT-IR) spectroscopy examines the twisting, bending, rotating, and vibrational motions of molecules. The spectra provide information that can be used to determine the presence or absence of a protein and give information regarding its conformation on the surface. Low-angle X-ray reflectometry may be used to determine the variations in electron density at an interface and allows resolution of packing differences in layers. Radioactive isotope labeling can be used to quantify the amount of protein adsorbed by ionization detection (Geiger counter) or liquid scintillation. See, for example, Y. S. Lin, V. Hlady and J. Janatova—Biomaterials, 13, (1992), p. 497. Solute depletion measures the amount of protein in solution before or after exposure to a surface. Scanning force microscopy uses a probe tip with a known position to characterize a surface species. The probe tip may be coated with specific molecules to determine chemical and physical interactions with a surface. See, for example, J. N. Lin, B. Drake, A. S. Lea, P. K. Hansma, and J. D. Andrade-Langmuir, 6, (1990), p. 509. Fluorescence emission spectroscopy measures the inherent fluorescence of a molecule or the fluorescence of a fluorescent label on a molecule. Proteins may be fluorescently labeled and detected using fluorimeters. See, for example, V. Hlady, Applied Spectroscopy 1991, 45, 246 and D. J. Sbrich and R. E. Imhof in Topics in Fluorescence Spectroscopy, J. R. Lakowicz Ed., Plenum, New York, (1991), p. 1, both of which are incorporated by reference herein. Circular dichroism measures the magnitude of polarized light rotation and detects the presence or absence of proteins. See, for example, C. R. McMillin and A. G. Walton—J. Colloid Interface Sci., 84, (1974), p. 345. Raman scattering is complimentary to infrared and measures the vibrational spectrum of molecules that undergo change in polarizability. It is used to determine the presence or absence of specific molecules/functional groups. See, for example, T. M. Cotton in Surface and Interfacial Aspects of Biomedical Polymers, 2, J. D. Andrade Ed., Plenum Press, New York, (1985), p. 161. Enzyme-linked immunosorbent assay testing is widely used to determine protein adsorption but requires the binding of multiple species for the detection of protein adsorption, not ultimately measuring directly the adsorbed protein. See for example M. Balcells, D. Klee, M. Fabry, and H. Hocker in J. Colloid Interfac. Sci. 1999, 220,198-204. Radiolabeling of proteins is a direct method for the assessment of proteins directly attached to surfaces but requires significant and costly training and equipment to handle and assess radioactivity. See, for example, C. Morin, A. P. Hitchcock, R. M. Cornelius, J. L. Brash, S. G. Urquhart, A. Scholl, A. Doran in J. Electron Microscopy Rel. Phenomena 2004, 137, 785-794. In general, this invention is not limited in any way by the nature of the forces holding the protein molecules to the substrates.

Direct monitoring of protein adsorption in pharmaceutical packaging is very challenging. Most methods that attempt to monitor protein adsorption depend on monitoring the change in concentration or activity of the protein left in the solution after a given period of time, subtracting that amount from the starting concentration to arrive at the amount of protein adsorption. However, it is very difficult to distinguish adsorption on the surface from denaturation and agglomeration in the solution caused by interaction with the packaging material and how this affects detection sensitivity in terms of concentration or activity. The methods described above, even those that observe directly at protein adsorption to a surface, are not amenable to studying true pharmaceutical packaging surfaces. For instance, the surface chemistry of glass vials and syringes change as a function glass type, glass manufacturer, and converting process. An unmet need of the pharmaceutical packaging industry is for an assay that can be used to directly quantify the adsorption of drugs to the pharmaceutically relevant surfaces of interest. The invention disclosed below describes a method for the direct quantification of drug adsorption to pharmaceutically relevant packaging surfaces that can be adapted to any pharmaceutical relevant packaging surface of interest with minor modifications. Upon further study of the specification and appended claims, further objects and advantages of this invention will become apparent to those skilled in the art.

### BRIEF DESCRIPTION OF THE DRAWINGS

Various features and attendant advantages of the present invention will be more fully appreciated as the same becomes better understood when considered in conjunction with the accompanying drawings, in which like reference characters designate the same or similar parts throughout the several views, and wherein:

FIG. 1: Direct vial assay method for the quantification of protein adsorption to pharmaceutically relevant surfaces via fluorescence spectroscopy on adsorbed protein aliquoted onto a partitioned glass surface.

FIG. 2: Calibration curve of Cy3 labeled fibrinogen as a function of fluorescence.

FIG. 3: Direct quantification of fibrinogen adsorption to four different surfaces.

FIG. 4: Theoretical cost savings for 2 or 6 μg protein adsorption/vial for low-concentration protein drugs.

FIG. 5: Direct quantification of histone III adsorption to nine different material/surface combinations.

FIG. 6: Direct quantification of insulin adsorption to nine different material/surface combinations.

As used herein, the terms “drug solution” and “drug” are interchangeably used and refers to a particular drug of interest in the presence of (typically) an aqueous buffered solution that may contain various additives. Typical drug solutions to be tested are derived from pharmaceutically relevant moieties such as cells, tissues, and derivatives thereof. Among the drugs are included any polyaminoacid chain, peptides, protein fragments and different types of proteins (e.g., structural, membrane, enzymes, antigens, monoclonal antibodies, polyclonal antibodies, ligands, receptors) produced naturally or recombinantly, as well as the derivatives of these compounds, etc. Specific protein drugs include antibodies (e.g. Remicade and ReoPro from Centocor; Herceptin from Genentech; Mylotarg from Wyeth, Synagis from MedImmune), enzymes (e.g. Pulmozyme from Genentech; Cerezyme from Genzyme), recombinant hormones (e.g., Protropin from Genentech, Novolin from Zymogenetics, Humulin from Lilly), recombinant interferon (e.g., Actimmune from InterMune Pharmaceutical; Avonex from Biogenldec, Betaseron from Chiron; Infergen from Amgen; Intron A from Schering-Plough; Roferon from Hoffman-La Roche), recombinant blood clotting cascade factors ( e.g., TNKase from Genentech; Retavase from Centocor; Refacto from Genetics Institute; Kogenate from Bayer) and recombinant erythropoietin (e.g., Epogen from Amgen; Procrit from J&J), and vaccines (e.g., Engerix-B from GSK; Recombivax HB from Merck & Co.).

Drugs can be labeled with various means conventionally known to one skilled in the art. A drug can be labeled with a radiolabel, a fluorescent label, a luminescent label or an enzymatic label. Preferably, the label is fluorescent.

One aspect of the invention is an assay method for the determination of drug adsorption to commercially available pharmaceutical packaging surfaces and test surfaces under development. Pharmaceutical packaging surface materials include glass, polymers (polyethylene, polypropylene, polycarbonate, polyterphalate, cylic olefin copolymer, etc.), elastomers (rubbers, etc.), metals and alloys for vials, syringes, ampoules, cartridges, stoppers, etc. and components thereof. The assay consists of the following steps:
1) Drug of interest is labeled with a fluorescent chemical group and degree of incorporation of dye is quantified.
2) Calibration curves of various concentrations of the fluorescently labeled biomolecules are generated.
3) Fluorescently labeled drug solution is exposed to the pharmaceutical packaging surface of interest for a length of time ranging from minutes to years.
4) Fluorescently labeled drug solution is removed from pharmaceutical packaging surface of interest. This solution can be analyzed to determine the amount of drugs remaining in the solution. The activity of the drugs may also be assessed by other methodology to help determine the stability of the drug.
5) Pharmaceutical packaging surface of interest is washed to remove any loosely adhering drugs.
6) Pharmaceutical packaging surface of interest is exposed to a solution to remove strongly adhered/bound or adsorbed drugs.
7) Solution is removed from pharmaceutical packaging surface of interest. Aliquots of the solution, or aliquots of the solution first concentrated, are then applied to a sample holding surface for determination of biomolecular adsorption via fluorescence measurements.

The sample holding surface in a preferred embodiment may be a Type 1 flat glass surface. The sample holding surface in a more preferred embodiment may be a polymer such as polypropylene, polyethylene, cylic olefin copolymer or physically/chemically treated/coated versions thereof. The sample holding surface in a most preferred embodiment may be the actual pharmaceutical package, which would eliminate steps 6 and 7 above.

Fluorescently labeled molecules are conventional and can be purchased from Amersham Biosciences/GE Healthcare. Test biomolecules such as fibrinogen, insulin, histone, and IgG have been purchased from Sigma-Aldrich.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### EXAMPLES

### Example 1

Adsorption of Fibrinogen to Various Coated Type 1 Glass Surfaces

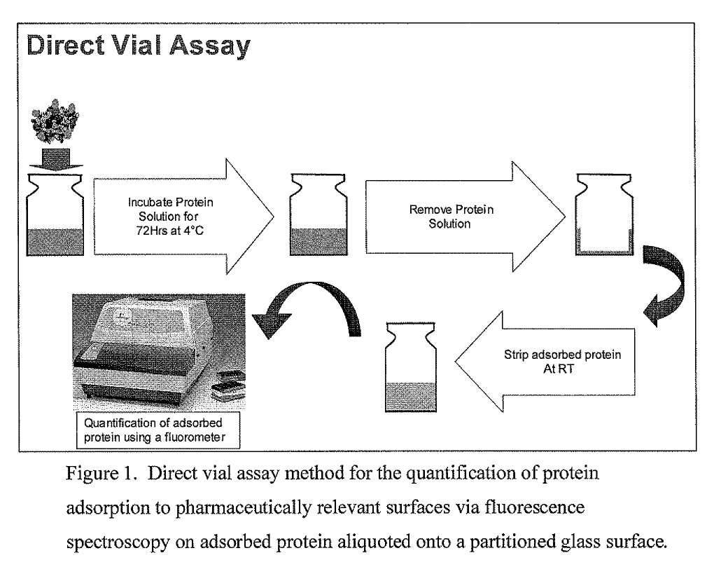

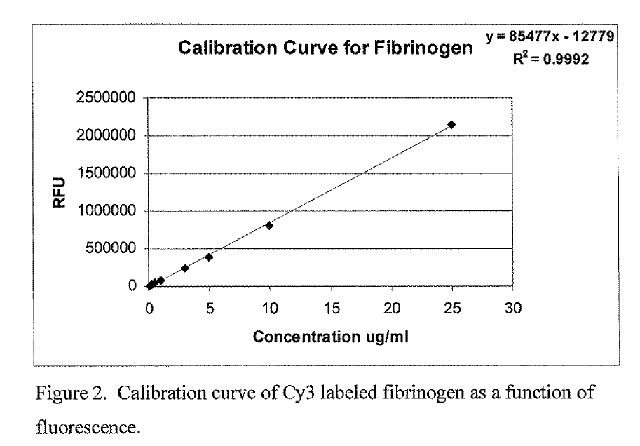

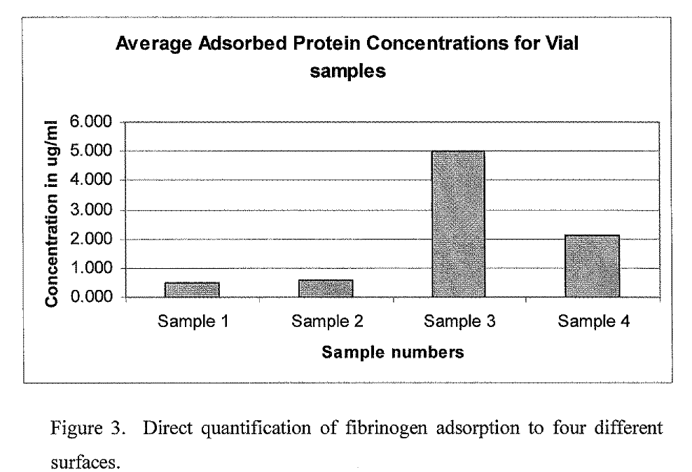

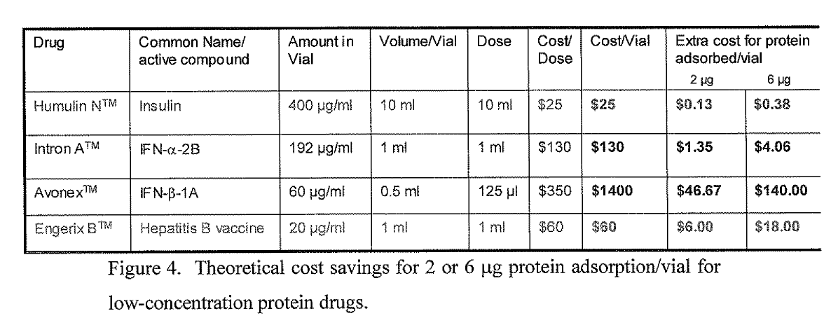

A pictorial representation of the assay method is shown in FIG. 1. Human fibrinogen, from Sigma (F3879; molecular weight 340,000 D; pI=5.4), is dissolved in 50 mM phosphate buffer (pH 7) to a 5 μg/mL concentration. Five different surface types are investigated. 12 replicates of sample surface 1 are prepared by plasma impulse chemical vapor deposition (PICVD) of tetraethyleneglycol dimethyl ether (TEGDE) with a pulse-time of 0.04 milliseconds onto Type 1 glass vials. 12 replicates of sample surface 2 are prepared by PICVD of TEGDE with a pulse-time of 0.15 milliseconds onto Type 1 glass vials. 12 replicates of sample surface 3 are prepared by PICVD of TEGDE with a pulse-time of 0.50 milliseconds onto Type 1 glass vials. The 12 replicates of sample surface 4 are uncoated Type 1 glass. Fibrinogen is labeled using a Cy3 antibody labeling kit from Amersham Biosciences. A calibration curve from the labeled fibrinogen is generated for the calculation of adsorbed proteins in FIG. 2. Protein solutions at 5 μg/mL are added to the vials for incubation at 4° C. for 72 hours. After incubation, the protein solution is removed from the vials and the vials are washed three times with water for injection. The vials are then exposed to a NaOH/sodiumdodecylsulfate solution for 90 minutes. The solution from each vial is then aliquoted in 50 μL volumes into wells on a glass slide. The slides are allowed to air-dry and scanned on an Axon 4000B laser scanner at 100% laser power 10-micron resolution with a gain of 400 in the Cy3 channel. The results are shown in FIG. 3. The results clearly indicate a quantifiable difference in protein adsorption between various sample surfaces. This experiment can also be conducted with a modification, that is, instead of incubating a labeled drug one can take an unlabeled drug, incubate, wash, then label the remaining adsorbed drug with a fluorescent moiety, followed either by direct analysis of the adsorbed drug on the surface or removal of the adsorbed drug and its subsequent analysis by fluorescence spectroscopy. The information gleaned from an assay of this type could lead to a cost reduction for drugs by providing information about drug adsorption that can be used to select packaging to minimize drug loss from drug/surface interaction effects. Shown in FIG. 4 are the hypothetical cost result savings for four low-concentration protein drugs assuming either a 2 or 6 μg adsorption of protein to packaging surface.

### Example 2

Adsorption of Histone III to Various Coated Surfaces.

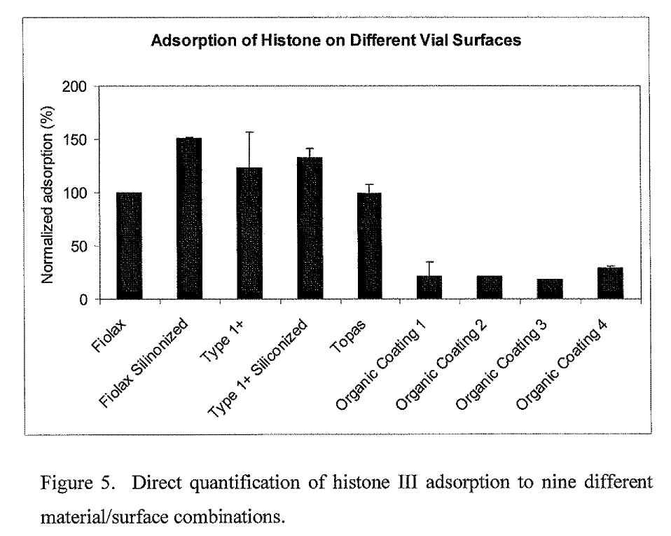

Calf thalamus histone III, from Sigma (H5505; molecular weight 293,000 D; pI=11.3), is dissolved in 50 mM phosphate buffer (pH 7) to a 10 μg/mL concentration. Nine different material/surface types are investigated. 3 replicates per sample surface are investigated. The data are normalized to the control surface, in this case a Type 1 vial. Sample surface 1 is a Type 1 vial. Sample surface 2 is a siliconized Type 1 vial. Sample surface 3 is a Schott Type 1+vial. Sample surface 4 is a siliconized Schott Type 1+vial. Sample surface 5 is a cylic olefin copolymer vial. Sample surface 6 is a Type 1 vial with an organic coating. Sample surface 7 is a Type 1 vial with a different organic coating. Sample surface 8 is a cylic olefin copolymer vial with the second organic coating. Sample surface 9 is a Schott Type 1+vial with the second organic coating. Histone III is labeled using a Cy3 labeling kit from Amersham Biosciences. Protein solutions at 10 μg/mL are added to the vials for incubation at 4° C. for 72 hours. After incubation, the protein solution is removed from the vials and the vials are washed three times with water for injection. The vials are then exposed to a NaOH/sodiumdodecylsulfate solution for 90 minutes. The solution from each vial is then aliquoted in 50 μL volumes into wells on a glass slide. The slides are allowed to air-dry and scanned on an Axon 4000B laser scanner at 100% laser power 10-micron resolution with a gain of 400 in the Cy3 channel. The results are shown in FIG. 5, normalized as percent adsorption compared to control samples of Type 1 glass vials. The results clearly indicate a quantifiable difference in protein adsorption between various sample surfaces. This experiment can also be conducted with a modification, that is, instead of incubating a labeled drug one can take an unlabeled drug, incubate, wash, then label the remaining adsorbed drug with a fluorescent moiety, followed either by direct analysis of the adsorbed protein on the surface or removal of the adsorbed drug and its subsequent analysis by fluorescence spectroscopy

### Example 3:

Adsorption of Insulin to Various Coated Surfaces.

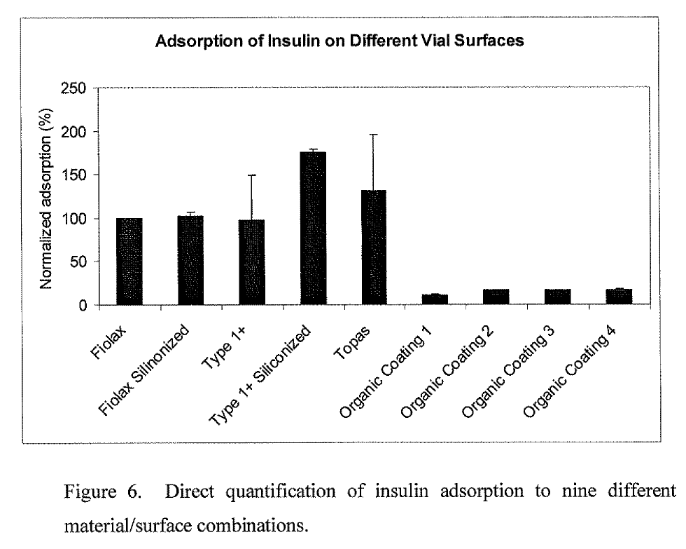

Porcine insulin, from Sigma (15523; molecular weight 5,800 D; pI=7.0), is dissolved in 50 mM phosphate buffer (pH 7) to a 10 μg/mL concentration. Nine different material/surface types are investigated. 3 replicates per sample surface are investigated. The data are normalized to the control surface, in this case a Type 1 vial. Sample surface 1 is a Type 1 vial. Sample surface 2 is a siliconized Type 1 vial. Sample surface 3 is a Schott Type 1+ vial. Sample surface 4 is a siliconized Schott Type 1+ vial. Sample surface 5 is a cylic olefin copolymer vial. Sample surface 6 is a Type 1 vial with an organic coating. Sample surface 7 is a Type 1 vial with a different organic coating. Sample surface 8 is a cylic olefin copolymer vial with the second organic coating. Sample surface 9 is a Schott Type 1+ vial with the second organic coating. Histone III is labeled using a Cy3 labeling kit from Amersham Biosciences. Protein solutions at 10 μg/mL are added to the vials for incubation at 4° C. for 72 hours. After incubation, the protein solution is removed from the vials and the vials are washed three times with water for injection. The vials are then exposed to a NaOH/sodiumdodecylsulfate solution for 90 minutes. The solution from each vial is then aliquoted in 50 μL volumes into wells on a glass slide. The slides are allowed to air-dry and scanned on an Axon 4000B laser scanner at 100% laser power 10 micron resolution with a gain of 400 in the Cy3 channel. The results are shown in FIG. 6. The results clearly indicate a quantifiable difference in protein adsorption between various sample surfaces. This experiment can also be conducted with a modification, that is, instead of incubating a labeled drug one can take an unlabeled drug, incubate, wash, then label the remaining adsorbed drug with a fluorescent moiety, followed either by direct analysis of the adsorbed drug on the surface or removal of the adsorbed drug and subsequent analysis by fluorescence spectroscopy.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of U.S. Provisional Application Ser. No. 60/845,534, filed Sep. 19, 2006, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

We claim:

1. A method of determining the adsorption of a drug to a pharmaceutical packaging surface comprising:
a) exposing a pharmaceutical packaging surface to a labeled or unlabeled drug,
b) washing the pharmaceutical packaging surface,
c) exposing the pharmaceutical packaging surface to an agent capable of removing bound drug, and
d) quantifying the amount of bound drug removed.

2. A method according to claim 1, wherein said drug is labeled with a radiolabel, a fluorescent label, a luminescent label or an enzymatic label.

3. A method according to claim 2, wherein calibration curves of various concentrations of fluorescently labeled drugs are generated.

4. A method according to claim 1, wherein said quantification of drug adsorption is conducted via spectroscopic analytical methods such as fluorescence spectroscopy, ultra-violet/visible/infra-red spectroscopy, mass spectroscopy, ellipsometry, and high pressure liquid chromatography.

5. A method according to claim 1, wherein said pharmaceutical packaging surface is exposed to a fluorescently labeled or unlabeled drug solution for a length of time ranging from seconds to years.

6. A method according to claim 1, wherein said drug is a protein or a peptide.

7. A method according to claim 1, wherein said pharmaceutical packaging surface of interest is a glass, polymer, cyclic olefin copolymer, elastomer, rubber, metal, or alloy.

8. A method according to claim 7, wherein said pharmaceutical packaging surface of interest further comprises one or more coatings.

9. A method according to claim 8, wherein said coatings are from the group of barrier, hydrophobic, hydrophilic, inorganic, organic, protein deterring, lubricious.

10. A method according to claim 8, wherein said coating is a silicon oxide or a polyether.

11. A method capable of determining the adsorption of a drug to a pharmaceutical packaging surface comprising:
a) exposing a pharmaceutical packaging surface to a drug solution,
b) removing said drug solution from the pharmaceutical packaging,
c) washing the pharmaceutical packaging surface,
d) exposing the pharmaceutical packaging surface to a fluorescent dye solution capable of binding to drug which is bound to the pharmaceutical packaging surface,
e) removing fluorescently labeled bound drug solution from the pharmaceutical packaging and
f) quantifying the amount of fluorescently labeled bound drug.

12. A method according to claim 11, wherein said aliquots of the fluorescent dye solution in step f are diluted or concentrated prior to quantifying.

13. A method according to claim 11, wherein said quantification of drug adsorption is conducted via spectroscopic analytical methods such as fluorescence spectroscopy, ultra-violet/visible/infra-red spectroscopy, mass spectroscopy, ellipsometry, and high pressure liquid chromatography.

14. A method according to claim 11, wherein said pharmaceutical packaging surface is exposed to the fluorescently labeled or unlabeled drug solution for a length of time ranging from seconds to years.

15. A method according to claim 11, wherein said drug is a protein or a peptide.

16. A method according to claim 11, wherein said pharmaceutical packaging surface of interest is a glass, polymer, cyclic olefin copolymer, elastomer, rubber, metal, or alloy.

17. A method according to claim 16, wherein said pharmaceutical packaging surface of interest further comprises one or more coatings.

18. A method according to claim 17, wherein said coatings are from the group of barrier, hydrophobic, hydrophilic, inorganic, organic, protein deterring, lubricious.

19. A method according to claim 17, wherein said coating is a silicon oxide or a polyether.

20. A method according to claim 1, wherein said pharmaceutical surface of interest is part of a syringe, an ampoule, a cartridge, a vial, an injector, or an intravenous drug delivery container.

21. A method according to claim 17, wherein said pharmaceutical surface is part of a syringe, an ampoule, a cartridge, a vial, an injector, or an intravenous drug delivery container.

22. An assay method capable of determining the adsorption of drugs to pharmaceutical packaging surfaces comprising:
a) labeling a drug with a fluorescent dye,
b) exposing a pharmaceutical packaging surface to said fluorescently labeled drug solution,
d) removing said fluorescently labeled drug solution from the pharmaceutical packaging,
e) washing the pharmaceutical packaging surface,
f) directly analyzing the pharmaceutical packaging surface of interest to quantify drug adsorption.

23. An assay method capable of determining the adsorption of drugs to pharmaceutical packaging surfaces comprising:
a) exposing a pharmaceutical packaging surface to a drug solution,
b) removing said drug solution from the pharmaceutical packaging,
c) washing the pharmaceutical packaging surface,
d) exposing the pharmaceutical packaging surface to a fluorescent dye solution capable of binding to drug which is bound to the pharmaceutical packaging surface,
e) removing the fluorescent dye solution from the pharmaceutical packaging surface and washing the pharmaceutical packaging,
f) directly analyzing the pharmaceutical packaging surface and quantifying the amount of drug adsorption.

24. A method according to claim 11, wherein aliquots of the fluorescent dye solution in step f are diluted or concentrated prior to quantifying.
